(19) United States
(12) Patent Application Publication
(10) Pub. No.: US 2007/0244000 A1
(43) Pub. Date: Oct. 18, 2007

(54) Producing olefin product from syngas

(76) Inventors: Michel Molinier, Houston, TX (US); James R. Lattner, LaPorte, TX (US); Matthew James Vincent, Baytown, TX (US); Kun Wang, Bridgewater, NJ (US)

Correspondence Address: ExxonMobil Chemical Company;Law Technology, P.O. Box 2149, Baytown, TX, 77522-2149, US

(21) Appl. No.: 11/711,279

(22) Filed: Feb. 26, 2007

Related U.S. Application Data
(60) Provisional application No. 60/791,736, filed on Apr. 13, 2006.

(51) Int. Cl. B01J 23/00; C07C 6/00

(52) U.S. Cl. 502/300; 585/327

(57) ABSTRACT

This invention is directed to the production of olefin product high in ethylene and propylene content using synthesis gas (syngas) as a feed. The syngas is converted to an intermediate composition high in methanol and dimethyl ether using a catalyst of at least two catalyst components, the first including at least one metal oxide and the second including at least one molecular sieve. The intermediate composition is then contacted with an olefin forming catalyst to form the olefin product.

### CROSS REFERENCE TO RELATED APPLICATIONS

This claims the benefit of and priority from U.S. Ser. No. 60/791,736, filed Apr. 13, 2006. The above application is fully incorporated herein by reference.

### FIELD OF THE INVENTION

This invention is directed to the production of olefin, particularly ethylene and propylene, from syngas. The production involves a catalyst for making a methanol and dimethyl ether composition that can be readily converted to olefin, and a method for making the catalyst.

### BACKGROUND OF THE INVENTION

Ethylene and propylene are chemical compounds that are used quite extensively as feed components in the manufacture of a fairly wide array of more complex chemical compositions. For example, ethylene is predominantly used as a feed compound in the production of low and high density polyethylene products. Approximately 60% of world ethylene consumption goes into making polyethylene for such products as plastic films, containers, and coatings. Other uses include the production of vinyl chloride, ethylene oxide, ethylbenzene, and alcohols. Presently, about 90% of the ethylene is produced by steam cracking petroleum-based feedstocks such as light paraffin, naphtha, and gas oil.

About 55% of the world consumption of propylene is directed to the production of polypropylene. Other important end products include acrylonitrile for acrylic and nylon fibers, and propylene oxide for polyurethane foams. About two-thirds of the propylene is produced from steam cracking petroleum feedstock, and the remaining third as a by-product of FCC gasoline refining.

A potential alternative to producing ethylene and propylene from petroleum-based feedstocks is to use methanol. Methanol is typically produced from synthesis gas, and synthesis gas is typically derived from the reforming of natural gas.

U.S. Pat. No. 4,499,327 (Kaiser) discloses making olefins from methanol using a variety of SAPO molecular sieve catalysts. The advantage of using SAPO based catalysts, particularly SAPO-34 based catalysts, is that such catalysts produce a substantially large amount of ethylene and propylene relative to other oxygenated hydrocarbons, e.g., alcohols, ethers, etc.

U.S. Pat. No. 5,085,762 (Absil) discloses a process of catalytically cracking hydrocarbons, and for specifically reducing the emission of noxious nitrogen oxides in the flue gas in FCC processes, by utilizing a copper-loaded zeolite catalyst, preferably MCM-22, made by ion exchange or impregnation. Although the processes disclosed in this patent do yield what are broadly characterized as hydrocarbon products, neither the reactants nor the products have similar compositions to those in the processes according to the invention below.

U.S. Pat. No. 6,518,475 (Xu) discloses a process of increasing ethylene selectivity in the conversion of a methanol-based feed. The method includes contacting a silicoaluminophosphate molecular sieve catalyst with a methanol composition that contains from about 1% to about 15% by weight acetone, and separating the ethylene and propylene from the olefin product. The use of the particular feed composition increases the amount of ethylene produced relative to that when pure methanol is used as the feed.

U.S. Patent Publication No. 2004/0116757 (Van Egmond) discloses a methanol-based composition, a method of making the composition, and a method of using the composition as a feedstock. The methanol composition contains methanol as a primary compound, but also includes one or more other alcohols such as ethanol and/or one or more aldehyde compounds. The methanol composition serves as a particularly desirable feed stream for use in the manufacture of olefins such as ethylene and propylene. Such feed streams result in increased production of ethylene or in the increased production of both ethylene and propylene.

U.S. Patent Publication No. 2005/0101815 (Xu) is directed to a process for converting oxygenates to olefins and olefins to polyolefins. The process includes a step of pretreating molecular sieve used in the conversion of oxygenate to olefin with a dimethyl ether composition. Fresh or regenerated molecular sieve, which is low in carbon content, is contacted or pretreated with the dimethyl ether composition to form a hydrocarbon co-catalyst within the pore structure of the molecular sieve, and the pretreated molecular sieve containing the co-catalyst is used to convert oxygenate to a lighter olefin product.

Although alcohol streams containing various alcohols and other oxygenates such as dimethyl ether are desirable for use as feed streams in the production of olefins such as ethylene and propylene, simpler processes are needed. In particular, processes that reduce the complexity of forming olefins from syngas are particularly desirable.

### SUMMARY OF THE INVENTION

This invention provides a methanol and dimethyl ether forming catalyst, a method of making the catalyst, and a process for using the catalyst to make an olefin product. The methanol and dimethyl ether that is formed can be readily converted to olefin that is particularly high in ethylene and propylene content.

According to one aspect of the invention, there is provided a methanol and dimethyl ether forming catalyst comprising a mixture of at least two catalyst components. The first of the catalyst components includes at least one oxide of at least one element selected from the group consisting of copper, silver, zinc, boron, magnesium, aluminum, vanadium, chromium, manganese, gallium, palladium, osmium, and zirconium, and the second of the catalyst components includes at least one MCM or SAPO molecular sieve.

Another aspect of the invention provides for a method of making a methanol and dimethyl ether forming catalyst. The method includes a step of mixing together a liquid and at least a first and second catalyst component to form a slurry. The first catalyst component contains at least one oxide of at least one element selected from the group consisting of copper, silver, zinc, boron, magnesium, aluminum, vanadium, chromium, manganese, gallium, palladium, osmium, and zirconium, and the second catalyst component contains at least one MCM or SAPO molecular sieve. The slurry is then dried to form the methanol and dimethyl ether forming catalyst.

The invention further provides a process for producing olefin product from syngas. The process includes a step of contacting a methanol and dimethyl ether forming catalyst with syngas to form a composition containing methanol and dimethyl ether. The catalyst comprises a first catalyst component that includes an oxide of at least one element selected from the group consisting of copper, silver, zinc, boron, magnesium, aluminum, vanadium, chromium, manganese, gallium, palladium, osmium, and zirconium. A second catalyst component is also provided, which includes at least one MCM or SAPO molecular sieve. The composition is contacted with an olefin forming catalyst to form the olefin product.

In one embodiment of the invention, the first of the catalyst components is a copper-based catalyst that includes at least one oxide of at least one element selected from the group consisting of silver, zinc, boron, magnesium, aluminum, vanadium, chromium, manganese, gallium, palladium, osmium, and zirconium. Preferably, the first of the catalyst components is a copper-based catalyst that includes at least one oxide of at least one element selected from the group consisting of zinc, magnesium, aluminum, chromium, manganese, and zirconium. More preferably, the first of the catalyst components is a copper-based catalyst that includes zinc oxide, manganese oxide, or a combination thereof.

In another embodiment of the invention, the molecular sieve is an intermediate or large pore MCM or SAPO molecular sieve. Preferably, the molecular sieve is an intermediate pore MCM or SAPO molecular sieve.

In yet another embodiment, the second of the catalyst components includes at least one MCM or SAPO molecular sieve selected from the group consisting of MCM-22, MCM-36, MCM-49, MCM-56, MCM-68, SAPO-11, SAPO-31, and SAPO-41. Preferably, the second of the catalyst components includes at least one MCM or SAPO molecular sieve selected from the group consisting of MCM-49, SAPO-11, and SAPO-41.

In another embodiment of the invention, the first of the catalyst components is a copper-based catalyst that includes at least one oxide of at least one element selected from the group consisting of zinc, magnesium, aluminum, chromium, manganese, and zirconium. Preferably, the first of the catalyst components is a copper-based catalyst that includes zinc oxide, manganese oxide, or a combination thereof.

### DETAILED DESCRIPTION OF THE INVENTION

### I. Manufacture of Olefin Product

This invention is directed to the production of olefin product high in ethylene and propylene content. The product is made using synthesis gas (syngas) as a feed. The syngas is converted to an intermediate composition high in methanol and dimethyl ether using a catalyst mixture. This intermediate is then sent to an olefin conversion unit as a feed stream and is converted to the olefin product. The olefin product is high in ethylene and propylene due to the methanol and dimethyl ether content of the intermediate feed composition. Such a feed composition is particularly beneficial in shifting the olefin product to a high concentration of ethylene.

The methanol and dimethyl ether forming catalyst of this invention comprises a mixture of at least two catalyst components. A first of the catalyst components includes at least one metal oxide. A second of the catalyst components includes at least one molecular sieve, preferably at least one zeolite molecular sieve, still more preferably at least one SAPO or MCM molecular sieve, and most preferably a large or medium pore SAPO or MCM molecular sieve.

The methanol and dimethyl ether forming catalyst can be made by mixing together a liquid and at least a first and second catalyst component to form a slurry. The slurry is then dried to form the methanol and dimethyl ether forming catalyst.

To make the olefin product, the syngas is contacted with the catalyst of the invention to form the methanol and dimethyl ether intermediate composition. The intermediate methanol and dimethyl ether composition is then contacted with an olefin forming catalyst to form the olefin product. The balance of the methanol and dimethyl ether composition in the intermediate is such that the olefin product that is produced is high in prime olefin (i.e., ethylene and propylene), and the olefin conversion catalyst used in that system has an appropriate balance of coke formed on the catalyst to maximize the selectivity to prime olefin.

### II. Synthesis Gas Production

### A. Methods of Making Synthesis Gas Feed

According to this invention, synthesis gas (syngas) is used as feed to make a mixed alcohol stream, and the mixed alcohol stream is then converted to olefin. Synthesis gas comprises carbon monoxide and hydrogen. Optionally, carbon dioxide and nitrogen are included.

Synthesis gas can be manufactured from a variety of carbon sources. Examples of such sources include biomass, natural gas, C₁-C₅ hydrocarbons, naphtha, heavy petroleum oils, or coke (i.e., coal). Preferably, the hydrocarbon feed stream comprises methane in an amount of at least about 50% by volume, more preferably at least about 70% by volume, most preferably at least about 80% by volume. In one embodiment of this invention natural gas is the preferred hydrocarbon feed source.

Any suitable syngas forming reactor or reaction system can be used in combination with the fluidized-bed reaction system of this invention. Examples of synthesis gas forming systems include partial oxidation, steam or CO₂ reforming, or some combination of these two chemistries.

### B. Steam Reforming to Make Syngas

In the catalytic steam reforming process, hydrocarbon feeds are converted to a mixture of H₂, CO, and CO₂ by reacting hydrocarbons with steam over a catalyst. This process involves the following reactions:

CH₄+H₂O⇄CO+3H₂ (1)

Or

CₙHₘ+nH₂O⇄nCO+[n+(m/2)]H₂ (2)

and

CO+H₂O⇄CO₂+H₂ (3) (shift reaction)

The reaction is carried out in the presence of a catalyst. Any conventional reforming type catalyst can be used. The catalyst used in the step of catalytic steam reforming comprises at least one active metal or metal oxide of Group 6 or Group 8-10 of the Periodic Table of the Elements. The Periodic Table of the Elements referred to herein is that from CRC Handbook of Chemistry and Physics, 82^{nd} Edition, 2001-2002, CRC Press LLC, which is incorporated herein by reference.

In one embodiment, the catalyst contains at least one Group 6 or Group 8-10 metal, or oxide thereof, having an atomic number of 28 or greater. Specific examples of reforming catalysts that can be used are nickel, nickel oxide, cobalt oxide, chromia, and molybdenum oxide. Optionally, the catalyst is employed with at least one promoter. Examples of promoters include alkali and rare earth promoters. Generally, promoted nickel oxide catalysts are preferred.

The amount of Group 6 or Group 8-10 metals in the catalyst can vary. Preferably, the catalyst includes from about 3 wt % to about 40 wt % of at least one Group 6 or Group 8-10 metal, based on total weight of the catalyst. Preferably, the catalyst includes from about 5 wt % to about 25 wt % of at least one Group 6 or Group 8-10 metal, based on total weight of the catalyst.

The reforming catalyst, optionally, contains one or more metals to suppress carbon deposition during steam reforming. Such metals are selected from the metals of Group 14 and Group 15 of the Periodic Table of the Elements. Preferred Group 14 and Group 15 metals include germanium, tin, lead, arsenic, antimony, and bismuth. Such metals are preferably included in the catalyst in an amount of from about 0.1 wt % to about 30 wt %, based on total weight of other metals (e.g., nickel) in the catalyst.

In a catalyst comprising nickel and/or cobalt there may also be present one or more platinum group metals, which are capable of increasing the activity of the nickel and/or cobalt and of decreasing the tendency to carbon lay-down when reacting steam with hydrocarbons higher than methane. The concentration of such platinum group metal is typically in the range 0.0005 to 0.1% w/w as metal, calculated as the whole catalyst unit. Further, the catalyst, especially in preferred forms, can contain a platinum group metal but no non-noble catalytic component. Such a catalyst is more suitable for the hydrocarbon steam reforming reaction than one containing a platinum group metal on a conventional support because a greater fraction of the active metal is accessible to the reacting gas. A typical content of platinum group metal when used alone is in the range 0.0005 to 0.5% w/w as metal, calculated on the whole catalytic unit.

In one embodiment, the reformer unit includes tubes which are packed with solid catalyst granules. Preferably, the solid catalyst granules comprise nickel or other catalytic agents deposited on a suitable inert carrier material. More preferably, the catalyst is NiO supported on calcium aluminate, alumina, spinel type magnesium aluminum oxide, or calcium aluminate titanate.

In yet another embodiment, both the hydrocarbon feed stream and the steam are preheated prior to entering the reformer. The hydrocarbon feedstock is preheated up to as high a temperature as is consistent with the avoiding of undesired pyrolysis or other heat deterioration. Since steam reforming is endothermic in nature, and since there are practical limits to the amount of heat that can be added by indirect heating in the reforming zones, preheating of the feed is desired to facilitate the attainment and maintenance of a suitable temperature within the reformer itself. Accordingly, it is desirable to preheat both the hydrocarbon feed and the steam to a temperature of at least 200° C.; preferably at least 400° C. The reforming reaction is generally carried out at a reformer temperature of from about 500° C. to about 1,200° C., preferably from about 800° C. to about 1,100° C., and more preferably from about 900° C. to about 1,050° C.

Gas hourly space velocity in the reformer should be sufficient for providing the desired CO to CO₂ balance in the synthesis gas. Preferably, the gas hourly space velocity (based on wet feed) is from about 3,000 per hour to about 10,000 per hour, more preferably from about 4,000 per hour to about 9,000 per hour, and most preferably from about 5,000 per hour to about 8,000 per hour.

Any conventional reformer can be used in the step of catalytic steam reforming. The use of a tubular reformer is preferred. Preferably, the hydrocarbon feed is passed to a tubular reformer together with steam, and the hydrocarbon and steam contact a steam reforming catalyst. In one embodiment, the steam reforming catalyst is disposed in a plurality of furnace tubes that are maintained at an elevated temperature by radiant heat transfer and/or by contact with combustion gases. Fuel, such as a portion of the hydrocarbon feed, is burned in the reformer furnace to externally heat the reformer tubes therein. See, for example, Kirk-Othmer, Encyclopedia of Chemical Technology, 3rd Ed., 1990, Vol. 12, p. 951; and Ullmann's Encyclopedia of Industrial Chemistry, 5th Ed., 1989, Vol. A-12, p. 186, the relevant portions of each being fully incorporated herein by reference.

The ratio of steam to hydrocarbon feed will vary depending on the overall conditions in the reformer. The amount of steam employed is influenced by the requirement of avoiding carbon deposition on the catalyst, and by the acceptable methane content of the effluent at the reforming conditions maintained. On this basis, the mole ratio of steam to hydrocarbon feed in the conventional primary reformer unit is preferably from about 1.5:1 to about 5:1, preferably from about 2:1 to about 4:1.

The hydrogen to carbon oxide ratio of the synthesis gas produced will vary depending on the overall conditions of the reformer. Preferably, the molar ratio of hydrogen to carbon oxide in the synthesis gas will range from about 1:1 to about 5:1. More preferably the molar ratio of hydrogen to carbon oxide will range from about 2:1 to about 3:1. Even more preferably the molar ratio of hydrogen to carbon oxide will range from about 2:1 to about 2.5:1. Most preferably the molar ratio of hydrogen to carbon oxide will range from about 2:1 to about 2.3:1.

Steam reforming is generally carried out at superatmospheric pressure. The specific operating pressure employed is influenced by the pressure requirements of the subsequent process in which the reformed gas mixture is to be employed. Although any superatmospheric pressure can be used in practicing the invention, pressures of from about 175 psig (1,308 kPa abs.) to about 1,100 psig (7,686 kPa abs.) are desirable. Preferably, steam reforming is carried out at a pressure of from about 300 psig (2,170 kPa abs.) to about 800 psig (5,687 kPa abs.), more preferably from about 350 psig (2,515 kPa abs.) to about 700 psig (4,928 kPa abs.).

### C. Partial Oxidation to Make Syngas

The invention further provides for the production of synthesis gas, or CO and H₂, by oxidative conversion (also referred to herein as partial oxidation) of hydrocarbon, particularly natural gas and C₁-C₅ hydrocarbons. According to the process, hydrocarbon is reacted with free-oxygen to form the CO and H₂. The process is carried out with or without a catalyst. The use of a catalyst is preferred, preferably with the catalyst containing at least one non-transition or transition metal oxides. The process is essentially exothermic, and is an incomplete combustion reaction, having the following general formula:

CₙHₘ+(n/2)O₂⇄nCO+(m/2)H₂ (4)

Non-catalytic partial oxidation of hydrocarbons to H₂, CO, and CO₂ is desirably used for producing syngas from heavy fuel oils, primarily in locations where natural gas or lighter hydrocarbons, including naphtha, are unavailable or uneconomical compared to the use of fuel oil or crude oil. The non-catalytic partial oxidation process is carried out by injecting preheated hydrocarbon, oxygen and steam through a burner into a closed combustion chamber. Preferably, the individual components are introduced at a burner where they meet in a diffusion flame, producing oxidation products and heat. In the combustion chamber, partial oxidation of the hydrocarbons generally occurs with less than stoichiometric oxygen at very high temperatures and pressures. Preferably, the components are preheated and pressurized to reduce reaction time. The process preferably occurs at a temperature of from about 1,350° C. to about 1,600° C., and at a pressure of from above atmospheric to about 150 atm.

Catalytic partial oxidation comprises passing a gaseous hydrocarbon mixture, and oxygen, preferably in the form of air, over reduced or unreduced catalysts. The reaction is optionally accompanied by the addition of water vapor (steam). When steam is added, the reaction is generally referred to as autothermal reduction. Autothermal reduction is both exothermic and endothermic as a result of adding both oxygen and water.

In the partial oxidation process, the catalyst comprises at least one transition element selected from the group consisting of Ni, Co, Pd, Ru, Rh, Ir, Pt, Os, and Fe. Preferably, the catalyst comprises at least one transition element selected from the group consisting of Pd, Pt, and Rh. In another embodiment, preferably the catalyst comprises at least one transition element selected from the group consisting of Ru, Rh, and Ir.

In one embodiment, the partial oxidation catalyst further comprises at least one metal selected from the group consisting of Ti, Zr, Hf, Y, Th, U, Zn, Cd, B, Al, TI, Si, Sn, Pb, P, Sb, Bi, Mg, Ca, Sr, Ba, Ga, V, and Sc. Also, optionally, included in the partial oxidation catalyst is at least one rare earth element selected from the group consisting of La, Ce, Pr, Nd, Pm, Sm, Eu, Gd, Th, Dy, Ho, Er, Tm, Yb, and Lu.

In another embodiment the catalyst employed in the process may comprise a wide range of catalytically active components, for example Pd, Pt, Rh, Ir, Os, Ru, Ni, Cr, Co, Ce, La, and mixtures thereof. Materials not normally considered to be catalytically active may also be employed as catalysts, for example refractory oxides such as cordierite, mullite, mullite aluminum titanate, zirconia spinels, and alumina.

In yet another embodiment, the catalyst is comprised of metals selected from those having atomic number 21 to 29, 40 to 47, and 72 to 79, the metals Sc, Ti V, Cr, Mn, Fe, Co, Ni, Cu, Zr, Nb, Mo, Tc, Ru, Rh, Pd, Ag, Hf, Ta, W, Re, Os, Ir, Pt, and Au. The preferred metals are those in Group 8 of the Periodic Table of the Elements, that is Fe, Os, Co, Re, Ir, Pd, Pt, Ni, and Ru.

In another embodiment, the partial oxidation catalyst comprises at least one transition or non-transition metal deposited on a monolith support. The monolith supports are preferably impregnated with a noble metal such as Pt, Pd, or Rh, or other transition metals such as Ni, Co, Cr, and the like. Desirably, these monolith supports are prepared from solid refractory or ceramic materials such as alumina, zirconia, magnesia, ceria, silica, titania, mixtures thereof, and the like. Mixed refractory oxides, that is refractory oxides comprising at least two cations, may also be employed as carrier materials for the catalyst.

In one embodiment, the catalyst is retained in the form of a fixed-arrangement. The fixed arrangement generally comprises a fixed bed of catalyst particles. Alternatively, the fixed arrangement comprises the catalyst in the form of a monolith structure. The fixed arrangement may consist of a single monolith structure or, alternatively, may comprise a number of separate monolith structures combined to form the fixed arrangement. A preferred monolith structure comprises a ceramic foam. Suitable ceramic foams for use in the process are available commercially.

In yet another embodiment, the feed comprises methane, and the feed is injected with oxygen into the partial oxidation reformer at a methane to oxygen (i.e., O₂) ratio of from about 1.2:1 to about 10:1. Preferably the feed and oxygen are injected into the reformer at a methane to oxygen ratio of from about 1.6:1 to about 8:1, more preferably from about 1.8:1 to about 4:1.

Water may or may not be added to the partial oxidation process. When added, the concentration of water injected into the reformer is not generally greater than about 65 mole %, based on total hydrocarbon and water feed content. Preferably, when water is added, it is added at a water to methane ratio of not greater than 3:1, preferably not greater than 2:1.

The catalyst may or may not be reduced before the catalytic reaction. In one embodiment, the catalyst is reduced and reduction is carried out by passing a gaseous mixture comprising hydrogen and inert gas (e.g., N₂, He, or Ar) over the catalyst in a fixed-bed reactor at a catalyst reduction pressure of from about 1 atm to about 5 atm, and a catalyst reduction temperature of from about 300° C. to about 700° C. Hydrogen gas is used as a reduction gas, preferably at a concentration of from about 1 mole % to about 100 mole %, based on total amount of reduction gas. Desirably, the reduction is further carried out at a space velocity of reducing gas mixture of from about 10³ cm³/g·hr to about 10⁵ cm³/g·hr for a period of from about 0.5 hour to about 20 hours.

In one embodiment, the partial oxidation catalyst is not reduced by hydrogen. When the catalyst is not reduced by hydrogen before the catalytic reaction, the reduction of the catalyst can be effected by passing the hydrocarbon feed and oxygen (or air) over the catalyst at a temperature in the range of from about 500° C. to about 900° C. for a period of from about 0.1 hour to about 10 hours.

In the partial oxidation process, carbon monoxide (CO) and hydrogen (H₂) are formed as major products, and water and carbon dioxide (CO₂) as minor products. The above-mentioned products, unconverted reactants (i.e., methane or natural gas and oxygen), and components of feed other than reactants are typically recovered as one or more gas product streams.

When water is added in the feed, the H₂:CO mole ratio in the product is increased by the shift reaction: CO+H₂O⇄H₂+CO₂. This reaction occurs simultaneously with the oxidative conversion of the hydrocarbon in the feed to CO and H₂ or synthesis gas. The hydrocarbon used as feed in the partial oxidation process is preferably in the gaseous phase when contacting the catalyst. The partial oxidation process is particularly suitable for the partial oxidation of methane, natural gas, associated gas or other sources of light hydrocarbons. In this respect, the term “light hydrocarbons” is a reference to hydrocarbons having from 1 to 5 carbon atoms. The process may be advantageously applied in the conversion of gas from naturally occurring reserves of methane which contain substantial amounts of carbon dioxide. In one embodiment, the hydrocarbon feed preferably contains from about 10 mole % to about 90 mole % methane, based on total feed content. More preferably, the hydrocarbon feed contains from about 20 mole % to about 80 mole % methane, based on total feed content. In another embodiment, the feed comprises methane in an amount of at least 50% by volume, more preferably at least 70% by volume, and most preferably at least 80% by volume.

In one embodiment of the invention, the hydrocarbon feedstock is contacted with the catalyst in a mixture with an oxygen-containing gas. Air is suitable for use as the oxygen-containing gas. Substantially pure oxygen as the oxygen-containing gas is preferred on occasions where there is a need to avoid handling large amounts of inert gas such as nitrogen. The feed, optionally, comprises steam.

In another embodiment of the invention, the hydrocarbon feedstock and the oxygen-containing gas are preferably present in the feed in such amounts as to give an oxygen-to-carbon ratio in the range of from about 0.3:1 to about 0.8:1, more preferably, in the range of from about 0.45:1 to about 0.75:1. References herein to the oxygen-to-carbon ratio refer to the ratio of oxygen in the form of oxygen molecules (O₂) to carbon atoms present in the hydrocarbon feedstock. Preferably, the oxygen-to-carbon ratio is in the range of from about 0.45:1 to about 0.65:1, with oxygen-to-carbon ratios in the region of the stoichiometric ratio of 0.5:1, that is ratios in the range of from about 0.45:1 to about 0.65:1, being more preferred. When steam is present in the feed, the steam-to-carbon ratio is not greater than about 3.0:1, more preferably not greater than about 2.0:1. The hydrocarbon feedstock, the oxygen-containing gas and steam, if present, are preferably well mixed prior to being contacted with the catalyst.

The partial oxidation process is operable over a wide range of pressures. For applications on a commercial scale, elevated pressures, that is pressures significantly above atmospheric pressure, are preferred. In one embodiment, the partial oxidation process is operated at pressures of greater than atmospheric up to about 150 bars. Preferably, the partial oxidation process is operated at a pressure in the range of from about 2 bars to about 125 bars, more preferably from about 5 bars to about 100 bars.

The partial oxidation process is also operable over a wide range of temperatures. At commercial scale, the feed is preferably contacted with the catalyst at high temperatures. In one embodiment, the feed mixture is contacted with the catalyst at a temperature in excess of 600° C. Preferably, the feed mixture is contacted with the catalyst at a temperature in the range of from about 600° C. to about 1,700° C., more preferably from about 800° C. to about 1,600° C. The feed mixture is preferably preheated prior to contacting the catalyst.

The feed is provided during the operation of the process at a suitable space velocity to form a substantial amount of CO in the product. In one embodiment, gas space velocities (expressed in normal liters of gas per kilogram of catalyst per hour) are in the range of from about 20,000 Ni/kg/hr to about 100,000,000 Nl/kg/hr, more preferably in the range of from about 50,000 Nl/kg/hr to about 50,000,000 Nl/kg/hr, and most preferably in the range of from about 500,000 Nl/kg/hr to about 30,000,000 Nl/kg/hr.

### D. Combination Syngas Processes

Combination reforming processes can also be incorporated into this invention. Examples of combination reforming processes include autothermal reforming and fixed-bed syngas generation. These processes involve a combination of gas phase partial oxidation and steam reforming chemistry.

The autothermal reforming process preferably comprises two synthesis gas generating processes, a primary oxidation process and a secondary steam reforming process. In one embodiment, a hydrocarbon feed stream is steam reformed in a tubular primary reformer by contacting the hydrocarbon and steam with a reforming catalyst to form a hydrogen and carbon monoxide-containing primary reformed gas, the carbon monoxide content of which is further increased in the secondary reformer. In one embodiment, the secondary reformer includes a cylindrical refractory lined vessel with a gas mixer, preferably in the form of a burner in the inlet portion of the vessel and a bed of nickel catalyst in the lower portion. In a more preferred embodiment, the exit gas from the primary reformer is mixed with air and residual hydrocarbons, and the mixed gas partial oxidized to carbon monoxides.

In another embodiment incorporating the autothermal reforming process, partial oxidation is carried out as the primary oxidizing process. Preferably, hydrocarbon feed, oxygen, and, optionally, steam, are heated and mixed at an outlet of a single large coaxial burner or injector which discharges into a gas phase partial oxidation zone. Oxygen is preferably supplied in an amount which is less than the amount required for complete combustion.

Upon reaction in the partial oxidation combustion zone, the gases flow from the primary reforming process into the secondary reforming process. In one embodiment, the gases are passed over a bed of steam reforming catalyst particles or a monolithic body, to complete steam reforming. Desirably, the entire hydrocarbon conversion is completed by a single reactor aided by internal combustion.

In an alternative embodiment of the invention, a fixed-bed syngas generation process is used to form synthesis gas. In the fixed-bed syngas generation process, hydrocarbon feed and oxygen or an oxygen-containing gas are introduced separately into a fluid catalyst bed. Preferably, the catalyst is comprised of nickel and supported primarily on alpha alumina.

The fixed-bed syngas generation process is carried out at conditions of elevated temperatures and pressures that favor the formation of hydrogen and carbon monoxide when, for example, methane is reacted with oxygen and steam. Preferably, temperatures are in excess of about 1,700° F. (927° C.), but not so high as to cause disintegration of the catalyst or the sticking of catalyst particles together. Preferably, temperatures range from about 1,750° F. (954° C.) to about 1,950° F. (1,066° C.), more preferably, from about 1,800° F. (982° C.) to about 1,850° F. (1,010° C.).

Pressure in the fixed-bed syngas generation process may range from atmospheric to about 40 atmospheres. In one embodiment, pressures of from about 20 atmospheres to about 30 atmospheres are preferred, which allows subsequent processes to proceed without intermediate compression of product gases.

In one embodiment of the invention, methane, steam, and oxygen are introduced into a fluid bed by separately injecting the methane and oxygen into the bed. Alternatively, each stream is diluted with steam as it enters the bed. Preferably, methane and steam are mixed at a methane to steam molar ratio of from about 1:1 to about 3:1, and more preferably from about 1.5:1 to about 2.5:1, and the methane and steam mixture is injected into the bed. Preferably, the molar ratio of oxygen to methane is from about 0.2:1 to about 1.0:1, more preferably from about 0.4:1 to about 0.6:1.

In another embodiment of the invention, the fluid bed process is used with a nickel based catalyst supported on alpha alumina. In another embodiment, silica is included in the support. The support is preferably comprised of at least 95 wt % alpha alumina, more preferably at least about 98% alpha alumina, based on total weight of the support.

### III. Syngas Feed

Synthesis gas (syngas) is used as a feedstock to form a composition comprising a major amount of methanol and dimethyl ether, which can then be used as a feed stream to form the olefin product high in ethylene and propylene. In one embodiment of the invention, the synthesis gas feed (including any recycle syngas recovered from the process itself, as well as fresh syngas) has a molar ratio of hydrogen (H₂) to carbon oxides (CO+CO₂) in the range of from about 0.5:1 to about 20:1, preferably in the range of from about 1:1 to about 10:1. In another embodiment, the synthesis gas has a molar ratio of hydrogen (H₂) to carbon monoxide (CO) of at least 2:1. Carbon dioxide is, optionally, present in an amount of not greater than 50% by weight, based on total weight of the synthesis gas, and preferably less than 20% by weight, more preferably less than 10% by weight.

The stoichiometric molar ratio [i.e., a molar ratio of (H₂+CO₂)/(CO+CO₂)] of the syngas should be sufficiently high so as to maintain a high yield of methanol and ethanol, but not so high as to reduce the volume productivity of both the methanol and dimethyl ether. In one embodiment, the synthesis gas used as feed for producing the methanol and dimethyl ether composition has a stoichiometric molar ratio of from about 1.0:1 to about 2.7:1, more preferably from about 1.5:1 to about 2.5:1, more preferably a stoichiometric molar ratio of from about 1.7:1 to about 2.5:1.

### IV. Converting Syngas to a Methanol and Dimethyl Ether Composition

The syngas is converted to a methanol and dimethyl ether intermediate composition by contacting the syngas with the methanol and dimethyl ether forming catalyst of this invention. The methanol and dimethyl ether forming catalyst of this invention is a mixture of at least two different catalyst components. As used herein, a mixture can be a mix of two or more individual components or a composite of at least two components that would be considered as a mechanical combination on a macroscale of two or more components that are solid in the finished state and differ in chemical nature. In one embodiment, the mixture is a product formed by a process comprising mixing together at least a first and second catalyst component to form a slurry, and drying the slurry.

At least one of the catalyst components of the catalyst of this invention includes at least one metal oxide. Preferably, at least one of the metal oxides is an oxide of at least one element selected from the group consisting of copper, silver, zinc, boron, magnesium, aluminum, vanadium, chromium, manganese, gallium, palladium, osmium, and zirconium. For convenience, the metal oxide component is also referred to herein as a first catalyst or catalyst component.

Preferably, the metal oxide or first catalyst is a copper-based oxide catalyst that includes an oxide of at least one element selected from the group consisting of silver, zinc, boron, magnesium, aluminum, vanadium, chromium, manganese, gallium, palladium, osmium, and zirconium. More preferably, the metal oxide catalyst is a copper-based oxide catalyst that includes an oxide of at least one element selected from the group consisting of zinc, magnesium, aluminum, chromium, manganese, and zirconium. Most preferably, the metal oxide catalyst is a copper-based oxide catalyst that includes zinc oxide, manganese oxide, or a combination thereof.

In one embodiment, generally when the metal includes copper, the active metal or metal oxide can contain more than 2.5 wt %, for example at least 2.8 wt % or at least 3 wt %, of the metal in the metal oxide component of the catalyst. Typically, the metal oxide portion of the catalyst contains no more than 92 wt % of the metal, in other embodiments no more than 90 wt %, no more than 85 wt %, no more than 80 wt %, no more than 75 wt %, or no more than 70 wt %.

At least one of the catalyst components of this invention includes a molecular sieve, preferably a crystalline molecular sieve, whether natural or synthetic. For convenience, the molecular sieve component is also referred to herein as a second catalyst or catalyst component.

Crystalline molecular sieves, both natural and synthetic, have been demonstrated to have catalytic properties for various types of hydrocarbon conversion processes. These molecular sieves are ordered, porous, crystalline material having a definite crystalline structure as determined by x-ray diffraction, a technique known to those of ordinary skill in the art. Within the crystalline structure, there are a large number of smaller cavities which may be interconnected by a number of still smaller channels or pores. The dimensions of these channels or pores are such as to allow for adsorption of molecules with certain dimensions, while rejecting those of large dimensions. The interstitial spaces or channels formed by the crystalline network enable molecular sieves such as crystalline silicates, aluminosilicates, crystalline silicoaluminophosphates, and crystalline aluminophosphates to be used as catalysts, which catalysts are particularly useful in this invention.

Within a pore of the crystalline molecular sieve, hydrocarbon conversion reactions that result in the formation of the alcohol product of this invention are governed by constraints imposed by the channel size of the molecular sieve. Reactant selectivity occurs when a fraction of the feedstock is too large to enter the pores to react; while product selectivity occurs when some of the products cannot leave the channels or do not subsequently react. Product distributions can also be altered by transition state selectivity in which certain reactions cannot occur because the reaction transition state is too large to form within the pores. Selectivity can also result from configuration constraints on diffusion where the dimensions of the molecule approach that of the pore system. Non-selective reactions on the surface of the molecular sieve, such as reactions on the surface acid sites of the molecular sieve, are generally not desirable as such reactions are not subject to the shape selective constraints imposed on those reactions occurring within the channels of the molecular sieve.

Zeolites are examples of crystalline microporous molecular sieves that are useful as the molecular sieve component of this invention. For purposes of this invention, zeolites are considered to be comprised of a lattice silica and, optionally, alumina combined with exchangeable cations such as alkali or alkaline earth metal ions. Although the term “zeolites” includes materials containing silica, and, optionally, alumina, the silica and alumina portions may be replaced in whole or in part with other oxides, if desired. For example, germanium oxide, titanium oxide, tin oxide, phosphorous oxide, and mixtures thereof can replace at least a portion of the silica. Likewise, boron oxide, iron oxide, gallium oxide, indium oxide, and mixtures thereof can replace at least a portion of the alumina. Accordingly, the terms “zeolite,” “zeolites,” and “zeolite material” refer not only to materials containing silicon, and, optionally, aluminum atoms in the crystalline lattice structure, but also materials which contain suitable replacement atoms for the silicon and/or aluminum, such as gallosilicates, borosilicates, silicoaluminophosphates (SAPO) and aluminophosphates (ALPO). The term “aluminosilicate zeolite” refers to zeolite materials consisting essentially of silicon and aluminum metal atoms in the crystalline lattice structure.

In general, zeolites are formed into aggregates such as pills, spheres, or extrudates by forming a slurry of the zeolite crystals in the presence of a binder and drying the resulting slurry. In one embodiment of this invention, the zeolite molecular sieve and the metal oxide are formed into a slurry and the slurry dried to form the alcohol forming catalyst of the invention. Binder and other materials are, optionally, used in forming the slurry. The binder materials used are preferably those that are resistant to high stress conditions such as temperature and mechanical stress conditions that occur in various hydrocarbon conversion processes. It is particularly preferred that the alcohol forming catalyst be resistant to mechanical attrition, that is, the formation of fines which are small particles, e.g., particles having a size of less than 20 microns. Examples of suitable binders that can be used include amorphous materials such as alumina, silica, titania, and various types of clays.

The terms “acidity,” “lower acidity,” “moderate acidity,” and “higher acidity” as generally applied to zeolites are understood by persons skilled in the art. The acidic properties of zeolites are generally well-known. However, a distinction can be made between acid strength and acid site density. Acid sites of a zeolite can be considered, for example, a Bronsted acid or a Lewis acid. The density of the acid sites and the number of acid sites are both used in determining the acidity of the zeolite. Factors directly influencing the acid strength are (i) the chemical composition of the zeolite framework, i.e., relative concentration and type of tetrahedral atoms, (ii) the concentration of the extra-framework cations and the resulting extra-framework species, (iii) the local structure of the zeolite, e.g., the pore size and the location, within the crystal or at/near the surface of the zeolite, and (iv) the pretreatment conditions and presence of co-adsorbed molecules. The amount of acidity is related to the degree of isomorphous substitution, provided such acidity is limited to the loss of acid sites for a pure SiO₂ composition. As used herein, the terms acidity,” “lower acidity” and “higher acidity” refers to the concentration of acid sites regardless of the strength of such acid sites which can be measured by ammonia absorption.

Zeolites suitable for use in the catalyst of this invention include any of the naturally occurring or synthetic crystalline zeolites. Examples of these zeolites include large pore zeolites, intermediate size pore zeolites, and small pore zeolites. Large and medium pore zeolites are particularly preferred. A large pore zeolite generally has a pore size of at least about 7 angstroms. For purposes of this invention, examples of large pore zeolites include MCM-9, MCM-41, MCM-41S, MCM-48, and SAPO-37. An intermediate pore size zeolite generally has a pore size from about 5 angstroms to about 7 angstroms. For purposes of this invention, examples of medium pore zeolites include MCM-22, MCM-36, MCM-49, MCM-56, MCM-68, SAPO-11, SAPO-31, and SAPO-41.

The methanol and dimethyl ether forming catalyst is typically made by mixing together one or more of the molecular sieves and one or more of the metal oxides, as well as a liquid solvent component (preferably water), to form a slurry. The components can be mixed in any order. Although other methods of forming a mixed catalyst composition exist, e.g., loading a molecular sieve with a metal or metal oxide through ion exchange or impregnation, these alternate methods are not preferred. Indeed, in a particular embodiment, one or more of the metal oxides are added to water and mixed together to form a first suspension or solution, and one or more molecular sieves are added to water to form a second solution or suspension. The solutions or suspensions are then mixed together to form a slurry, and the slurry is dried to form the catalyst.

The molecular sieve and one or more metal oxides are mixed together to form a slurry having a desired content. In one embodiment, molecular sieve(s) and metal oxide(s) are mixed together to form a slurry having a molecular sieve solids content of at least 5 wt %, based on total weight of the solids in the slurry mixture (i.e., excluding liquid). Preferably, molecular sieve(s) and metal oxide(s) are mixed together to form a slurry having a molecular sieve solids content of from 5 wt % to 90 wt %, more preferably from 10 wt % to 80 wt %, and most preferably from 20 wt % to 70 wt %, based on total weight of the solids in the slurry mixture.

The temperature at which the slurry is made can range. Examples of such conditions include temperatures ranging from 0° C. to 120° C., preferably of from 20° C. to 110° C., more preferably of from 40° C. to 100° C., most preferably of from 60° C. to 90° C.

In batch operation, the mixer for mixing the slurry can be operated for some duration to ensure proper mixing and viscosity. In one embodiment, mixer is in-tank operated for a period of at least 2 hours, preferably at least 4 hours, more preferably at least 5 hours, and most preferably at least 6 hours. In a preferred embodiment, mixing of slurry components is performed for not more than 150 hours, preferably not more than 120 hours, most preferably not more than 100 hours. Other preferred batch mixing conditions include mixing at a temperature of from 30° C. to 50° C. for a period of from 4 hours to 80 hours, preferably from 5 hours to 75 hours, more preferably of from 5.5 hours to 50 hours, most preferably of from 6 hours to 36 hours.

In one embodiment, the slurry of the molecular sieve and metal oxide is fed to a forming unit that produces a dried catalyst composition. Non-limiting examples of forming units include spray dryers, pelletizers, extruders, etc. Typically, the forming unit is maintained at a temperature sufficient to remove most of the liquid (e.g., water) from the slurry. In one embodiment, the slurry is dried at a temperature in the range of from about 100° C. to about 500° C., preferably from about 110° C. to about 500° C., and most preferably from about 120° C. to about 550° C., preferably in an environment such as air, nitrogen, helium, flue gas (combustion product lean in oxygen), or any combination thereof.

In one embodiment, the molecular sieve and metal oxide catalyst composition is further heated in air at a temperature of from about 50° C. to about 500° C. Heating is carried out for a period of time typically from 30 minutes to 15 hours, preferably from 1 hour to about 10 hours.

The conversion of syngas to the methanol and dimethyl ether intermediate can be accomplished over a wide range of temperatures. Lower to mid-temperature ranges are preferred. In one embodiment, the syngas is contacted with the catalysts at a temperature in the range of from about 150° C. to about 550° C., preferably in a range of from about 175° C. to about 450° C., more preferably in a range of from about 200° C. to about 400° C.

The syngas can also be converted to the intermediate composition over a wide range of pressures. In one embodiment, the syngas is contacted with the catalyst at a pressure in the range of from about 15 atmospheres to about 125 atmospheres, preferably in a range of from about 20 atmospheres to about 100 atmospheres, more preferably in a range of from about 25 atmospheres to about 75 atmospheres.

Gas hourly space velocities in converting the syngas to alcohol product can vary depending upon the type of reactor that is used. In one embodiment, gas hourly space velocity of flow of gas through the catalyst bed is in the range of from about 50 hr⁻¹ to about 50,000 hr⁻¹. Preferably, gas hourly space velocity of flow of gas through the catalyst bed is in the range of from about 250 hr⁻¹ to about 25,000 hr⁻¹, more preferably from about 500 hr⁻¹ to about 20,000 hr⁻¹.

The methanol and dimethyl ether intermediate composition of this invention can be used as feed for any conventional process. Examples of such uses include the manufacture of methyl tertiary butyl alcohol (MTBE) for use in reformulated gasolines and oxygenated fuels; the use of methanol as a fuel for fuel cells, use as feedstock to make olefins, and for use in making acetic acid and formaldehyde.

The intermediate product stream of this invention is particularly suited for conversion to olefins, particularly ethylene and/or propylene. The methanol product stream can be fed directly to an olefin conversion process or it can be transported in large quantities over great distances and converted to olefins.

According to this invention, the intermediate product can be produced in large scale quantities for conversion to olefins, which is of great advantage for further conversion of the olefins to polyolefins such as polyethylene and polypropylene. Advantageously, this invention allows for at least 100,000 metric tons of methanol product per year. Preferably, production is at least 500,000 metric tons per year, more preferably at least 1 million metric tons per year, and most preferably at least 2 million metric tons per year.

In one embodiment, the intermediate product stream of the invention is transported to a location geographically distinct from that where the composition was produced. Preferably, the intermediate methanol and dimethyl ether composition of this invention is loaded into a vessel, and the vessel is transported over a body of water to a storage facility. The composition can be easily transported at least 100, 500, or 1,000 miles or more. Once arriving at the storage facility, the composition is delivered to a storage tank. From the storage tank, the composition is ultimately sent to an olefin conversion unit for conversion to an olefin product. The composition is preferably, loaded onto a ship, with the ship able to contain at least 20,000 tons, preferably at least 40,000 tons, and more preferably at least 80,000 tons.

An advantage of being able to transport the methanol and dimethyl ether intermediate composition is that the units which produce the composition do not have to be located in close geographic proximity to the olefin conversion unit. This makes it possible to use remote gas reserves. These remote gas reserves would be used as feed for the intermediate composition manufacturing facility. The composition made at these remote sites can then be easily transported to a suitable location for conversion to olefins. Since olefins and polyolefins (i.e., plastics) demands are typically low at the remote gas sites, there will generally be a desire to transport methanol and dimethyl ether composition to high olefins and plastic demand areas. The composition can be routinely transported in vessels that are similar to those that transport crude oil and other fuels. Examples of locations of remote gas reserves include Africa, Australia, the Indian Ocean, China, and the Arabian Peninsula. Examples of locations of preferred sites to convert methanol and dimethyl ether to other products such as olefins include the U.S. Gulf Coast and Europe.

### V. Converting the Methanol and Dimethyl Ether Composition to Olefins

### A. General Process Description

In one embodiment of the invention, the methanol and dimethyl ether composition is converted to olefins by contacting the methanol and dimethyl ether composition with an olefin forming catalyst to form the olefin product. The olefin product is recovered, and water, which forms during the conversion of the oxygenates in the methanol to olefins, is removed. After removing the water, the olefins are separated into individual olefin streams, and each individual olefin stream is available for further processing.

### B. Description of Olefin Forming Catalyst

Any catalyst capable of converting oxygenate such as methanol and dimethyl ether to olefin can be used in this invention. Molecular sieve catalysts are preferred. Examples of such catalysts include zeolite as well as non-zeolite molecular sieves, and are of the large, medium, or small pore type. Non-limiting examples of these molecular sieves are the small pore molecular sieves, AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, and substituted forms thereof; the medium pore molecular sieves, AFO, AEL, EUO, HEU, FER, MEL, MFI, MTW, MTT, TON, and substituted forms thereof; and the large pore molecular sieves, EMT, FAU, and substituted forms thereof. Other molecular sieves include ANA, BEA, CFI, CLO, DON, GIS, LTL, MER, MOR, MWW, and SOD. Non-limiting examples of the preferred molecular sieves, particularly for converting an oxygenate containing feedstock into olefin(s), include AEL, AFY, BEA, CHA, EDI, FAU, FER, GIS, LTA, LTL, MER, MFI, MOR, MTT, MWW, TAM, and TON. In one preferred embodiment, the molecular sieve of the invention has an AEI topology or a CHA topology, or a combination thereof, most preferably a CHA topology.

In one embodiment, aluminophosphate (ALPO) molecular sieves, silicoaluminophosphate (SAPO) molecular sieves or a combination thereof is used. Preferred molecular sieves are SAPO molecular sieves, and metal substituted SAPO molecular sieves. In an embodiment, the metal is an alkali metal of Group IA of the Periodic Table of Elements, an alkaline earth metal of Group IIA of the Periodic Table of Elements, a rare earth metal of Group IIIB, including the Lanthanides: lanthanum, cerium, praseodymium, neodymium, samarium, europium, gadolinium, terbium, dysprosium, holmium, erbium, thulium, ytterbium and lutetium; and scandium or yttrium of the Periodic Table of Elements, a transition metal of Groups IVB, VB, VIIB, VIIB, VIIIB, and IB of the Periodic Table of Elements, or mixtures of any of these metal species. In one preferred embodiment, the metal is selected from the group consisting of Co, Cr, Cu, Fe, Ga, Ge, Mg, Mn, Ni, Sn, Ti, Zn, and Zr, and mixtures thereof. In another preferred embodiment, these metal atoms discussed above are inserted into the framework of a molecular sieve through a tetrahedral unit, such as [MeO₂], and carry a net charge depending on the valence state of the metal substituent. For example, in one embodiment, when the metal substituent has a valence state of +2, +3, +4, +5, or +6, the net charge of the tetrahedral unit is between −2 and +2.

Non-limiting examples of SAPO and ALPO molecular sieves used in the invention include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44 (U.S. Pat. No. 6,162,415), SAPO-47, SAPO-56, ALPO-5, ALPO-11, ALPO-18, ALPO-31, ALPO-34, ALPO-36, ALPO-37, ALPO-46, and metal containing molecular sieves thereof. The more preferred zeolite-type molecular sieves include one or a combination of SAPO-18, SAPO-34, SAPO-35, SAPO-44, SAPO-56, ALPO-18, and ALPO-34, even more preferably one or a combination of SAPO-18, SAPO-34, ALPO-34, and ALPO-18, and metal containing molecular sieves thereof, and most preferably one or a combination of SAPO-34 and ALPO-18, and metal containing molecular sieves thereof.

In an embodiment, the molecular sieve is an intergrowth material having two or more distinct phases of crystalline structures within one molecular sieve composition. In particular, intergrowth molecular sieves are described in the U.S. Pat. No. 6,812,372 and PCT WO 98/15496 published Apr. 16, 1998, both of which are fully incorporated herein by reference. In another embodiment, the molecular sieve comprises at least one intergrown phase of AEI and CHA framework types. For example, SAPO-18, ALPO-18, and RUW-18 have an AEI framework type, and SAPO-34 has a CHA framework type.

The molecular sieves are made or formulated into catalysts by combining the synthesized molecular sieves with a binder and/or a matrix material to form a molecular sieve catalyst composition or a formulated molecular sieve catalyst composition. This formulated molecular sieve catalyst composition is formed into useful shape and sized particles by conventional techniques such as spray drying, pelletizing, extrusion, and the like.

### C. General Conditions for Converting Alcohol to Olefins

According to the reaction process of this invention, the mixed alcohol stream is contacted with olefin forming catalyst to form an olefin product, particularly ethylene and propylene. The process for converting the oxygenate feedstock is, preferably, a continuous fluidized-bed process, and most preferably a continuous high velocity fluidized-bed process.

The reaction processes can take place in a variety of catalytic reactors such as hybrid reactors that have dense bed or fixed-bed reaction zones and/or fast fluidized-bed reaction zones coupled together, circulating fluidized-bed reactors, riser reactors, and the like. Suitable conventional reactor types are described in, for example, U.S. Pat. Nos. 4,076,796 and 6,287,522 (dual riser), and Fluidization Engineering, D. Kunii and O. Levenspiel, Robert E. Krieger Publishing Company, New York, N.Y. 1977.

One preferred reactor type is a riser reactor. These types of reactors are generally described in Riser Reactor, Fluidization and Fluid-Particle Systems, pp. 48 to 59, F. A. Zenz and D. F. Othmo, Reinhold Publishing Corporation, N.Y., 1960, and U.S. Pat. No. 6,166,282 (fast-fluidized-bed reactor).

In one embodiment of the invention, a fluidized-bed process or high velocity fluidized-bed process includes a reactor system, catalyst separation system, and a regeneration system. The reactor system preferably is a fluid bed reactor system. In one embodiment, the fluid bed reactor system has a first reaction zone within one or more riser reactors, and a second reaction zone within at least one catalyst separation vessel, preferably comprising one or more cyclones. In one embodiment, one or more riser reactors and catalyst separation vessel is contained within a single reactor vessel.

The average reaction temperature employed in the conversion process, specifically within the reactor, is of from about 250° C. to about 800° C. Preferably the average reaction temperature within the reactor is from about 250° C. to about 750° C.; more preferably, from about 300° C. to about 650° C.; yet more preferably from about 350° C. to about 600° C.; and most preferably from about 400° C. to about 500° C.

The pressure employed in the conversion process, specifically within the reactor, is not critical. The reaction pressure is based on the partial pressure of the feedstock exclusive of any diluent therein. Typically, the reaction pressure employed in the process is in the range of from about 0.1 kPaa to about 5 MPaa, preferably from about 5 kPaa to about 1 MPaa, and most preferably from about 20 kPaa to about 500 kPaa.

The weight hourly space velocity (WHSV), defined as the total weight of the feedstock excluding any diluents to the reaction zone per hour per weight of molecular sieve in the molecular sieve catalyst composition in the reaction zone, is maintained at a level sufficient to keep the catalyst composition in a fluidized state within a reactor. Typically, the WHSV ranges from about 1 hr⁻¹ to about 5000 hr⁻¹, preferably from about 2 hr⁻¹ to about 3000 hr⁻¹, more preferably from about 5 hr⁻¹ to about 1500 hr⁻¹, and most preferably from about 10 hr⁻¹ to about 1000 hr⁻¹. In one preferred embodiment, the WHSV is greater than 20 hr⁻¹, preferably the WHSV for conversion of a feedstock containing methanol and dimethyl ether is in the range of from about 20 hr⁻¹ to about 300 hr⁻¹.

The superficial gas velocity (SGV) of the feedstock, including diluent and reaction products within the reactor, is preferably sufficient to fluidize the molecular sieve catalyst composition within a reaction zone of the reactor. The SGV in the process, particularly within the reactor system, more particularly within a riser reactor, is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec.

### VI. Olefin Product Recovery and Use

In one embodiment, olefin product and other gases are withdrawn from the reactor and are passed through a recovery system. Any conventional recovery system, technique and/or sequence useful in separating olefin(s) and purifying olefin(s) from other gaseous components can be used in this invention. Examples of recovery systems include one or more or a combination of various separation, fractionation and/or distillation towers, columns, and splitters, and other associated equipment; for example, various condensers, heat exchangers, refrigeration systems or chill trains, compressors, knock-out drums or pots, pumps, and the like.

Non-limiting examples of distillation towers, columns, splitters or trains used alone or in combination include one or more of a demethanizer, preferably a high temperature demethanizer, a deethanizer, a depropanizer, preferably a wet depropanizer, a wash tower often referred to as a caustic wash tower and/or quench tower, absorbers, adsorbers, membranes, ethylene (C₂) splitter, propylene (C₃) splitter, butene (C₄) splitter, and the like.

Generally accompanying most recovery systems is the production, generation or accumulation of additional products, by-products and/or contaminants along with the preferred prime products. The preferred prime products, the light olefins, such as ethylene and propylene, are typically purified for use in derivative manufacturing processes such as polymerization processes.

The ethylene and propylene streams produced and recovered according to this invention can be polymerized to form plastic compositions, e.g., polyolefins, particularly polyethylene and polypropylene. Any process capable of forming polyethylene or polypropylene can be used. Catalytic processes are preferred. Particularly preferred are metallocene, Ziegler/Natta, aluminum oxide and acid catalytic systems. In general, these methods involve contacting the ethylene or propylene product with a polyolefin-forming catalyst at a pressure and temperature effective to form the polyolefin product.

In one embodiment of this invention, the ethylene or propylene product is contacted with a metallocene catalyst to form a polyolefin. Desirably, the polyolefin forming process is carried out at a temperature ranging between about 50° C. and about 320° C. The reaction can be carried out at low, medium, or high pressure, being anywhere within the range of about 1 bar to about 3200 bar. For processes carried out in solution, an inert diluent can be used. In this type of operation, it is desirable that the pressure be at a range of from about 10 bar to about 150 bar, and preferably at a temperature range of from about 120° C. to about 250° C. For gas phase processes, it is preferred that the temperature generally be within a range of about 60° C. to 120° C., and that the operating pressure be from about 5 bar to about 50 bar.

In addition to polyolefins, numerous other olefin derivatives may be formed from the ethylene, propylene and C₄+ olefins, particularly butylene, separated according to this invention. The olefins separated according to this invention can also be used in the manufacture of such compounds as aldehydes, acids such as C₂-C₁₃ mono carboxylic acids, alcohols such as C₂-C₁₂ mono alcohols, esters made from the C₂-C₁₂ mono carboxylic acids and the C₂-C₁₂ mono alcohols, linear alpha olefins, vinyl acetate, ethylene dicholoride and vinyl chloride, ethylbenzene, ethylene oxide, cumene, acrolein, allyl chloride, propylene oxide, acrylic acid, ethylene-propylene rubbers, and acrylonitrile, and trimers and dimers of ethylene and propylene. The C₄+ olefins, butylene, in particular, are particularly suited for the manufacture of aldehydes, acids, alcohols, esters made from C₅-C₁₃ mono carboxylic acids and C₅-C₁₃ mono alcohols and linear alpha olefins.

### VII. EXAMPLES

### Example 1 (Comparative Example)

This example describes the preparation of a methanol and dimethyl ether forming catalyst (A) having the following composition:

50% ZSM-5/50% (60% CuO/30% ZnO/10% Mn₂O₃ (nominal)

The following solutions were prepared:

2 M Cu: 35.4 g Cu(NO₃)₂*3H₂O were dissolved in 75 cc d.i. H₂O

2 M Zn: 22 g Zn(NO₃)₂*6H₂O were dissolved in 37 cc d.i. H₂O

2 M Mn: 4.5 g Mn(NO₃)₂*xH₂O were dissolved in 13 cc d.i. H₂O

2m (NH₄)₂CO₃: 72.1 g (NH₄)₂CO₃ were dissolved in 375 cc d.i. H₂O

A 2 liter flask was filled with a slurry made of 20 g zeolite H-ZSM-5 (H-MFI 45, Sud Chemie 03677-S) and 500 ml d.i. H₂O. The slurry was heated to 70° C. and stirred. The Cu, Zn, and Mn solutions were mixed to form a solution 1. The (NH₄)₂CO₃ solution was labeled as solution 2. While stirring the slurry and maintaining its temperature at 70° C., solutions 1 and 2 were added simultaneously to the water, with pH control during addition at 7.0. The reaction was considered complete when all of solution 1 had been added. The mixture was then kept at temperature and under stirring for another hour. The stirring was stopped and the mixture was cooled to room temperature. The precipitate was then filtered and thoroughly washed with warm d.i. H₂O (1500-2000 cc H₂O). The precipitate was finally dried overnight at 85° C.

The precipitate was calcined under air according to the following schedule:

1) 2 hours ramp from room temperature to 150° C.

2) 0.5 hour at 150° C.

3) 2 hours ramp from 150° C. to 350° C.

4) 3 hours at 350° C., then heating stopped

5) cool down to room temperature

### Example 2 (Comparative Example)

This example describes the preparation of a methanol and dimethyl ether forming catalyst (B) having the following composition:

60% CuO/30% ZnO/10% Mn₂O₃ (nominal)

The following solutions were prepared:

2 M Cu: 35.4 g Cu(NO₃)₂*3H₂O were dissolved in 75 cc d.i. H₂O

2 M Zn: 22.0 g Zn(NO₃)₂*6H₂O were dissolved in 37 cc d.i. H₂O

2 M Mn: 4.5 g Mn(NO₃)₂*xH₂O were dissolved in 13 cc d.i. H₂O

2 M Na₂CO₃: 93.0 g Na₂CO₃*H₂O were dissolved in 375 cc d.i. H₂O

A 2 liter flask was filled with 500 ml d.i. H₂O. The water was heated to 70° C. and stirred. The Cu, Zn, and Mn solutions were mixed to form a solution 1. The Na₂CO₃ solution was labeled as solution 2. While stirring the water and maintaining its temperature at 70° C., solutions 1 and 2 were added simultaneously to the water, with pH control during addition at 7.0. The reaction was considered complete when all of solution 1 had been added. The mixture was then kept at temperature and under stirring for another hour. The stirring was stopped and the mixture was cooled to room temperature. The precipitate was then filtered and thoroughly washed with warm d.i. H₂O (1500-2000 cc H₂O). The precipitate was finally dried overnight at 85° C.

The precipitate was calcined under air according to the following schedule:

1) 2 hours ramp from room temperature to 150° C.

2) 0.5 hour at 150° C.

3) 2 hours ramp from 150° C. to 350° C.

4) 3 hours at 350° C., then heating stopped

5) cool down to room temperature

### Example 3

This example describes the preparation of a methanol and dimethyl ether forming catalyst (C) having the following composition:

50% SAPO-11/50% (60% CuO/30% ZnO/10% Mn₂O₃ (nominal)

The following solutions were prepared:

2 M Cu: 18.2 g Cu(NO₃)₂*3H₂O were dissloved in 38 cc d.i. H₂O

2 M Zn: 11.0 g Zn(NO₃)₂*6H₂O were dissloved in 19 cc d.i. H₂O

2 M Mn: 2.3 g Mn(NO₃)₂*xH₂O were dissloved in 7 cc d.i. H₂O

2 M Na₂CO₃: 47.0 g Na₂CO₃*H₂O were dissloved in 188 cc d.i. H₂O

A 2 liter flask was filled with a slurry made of 10 g SAPO-11 (OLE 230 from UOP) and 500 ml d.i. H₂O. The slurry was heated to 70° C. and stirred. The Cu, Zn, and Mn solutions were mixed to form a solution 1. The Na₂CO₃ solution was labeled as solution 2. While stirring the slurry and maintaining its temperature at 70° C., solutions 1 and 2 were added simultaneously to the water, with pH control during addition at 7.0. The reaction was considered complete when all of solution 1 had been added. The mixture was then kept at temperature and under stirring for another hour. The stirring was stopped and the mixture cooled to room temperature. The precipitate was then filtered and thoroughly washed with warm d.i. H₂O (1500-2000 cc H₂O). The precipitate was finally dried overnight at 85° C.

The precipitate was calcined under air according to the following schedule:

1) 2 hours ramp from room temperature to 150° C.

2) 0.5 hour at 150° C.

3) 2 hours ramp from 150° C. to 350° C.

4) 3 hours at 350° C., then heating stopped

5) cool down to room temperature

### Example 4

This example describes the preparation of a methanol and dimethyl ether forming catalyst (D) having the following composition:

50% SAPO-41/50% (60% CuO/30% ZnO/10% Mn₂O₃) (nominal)

The following solutions were prepared:

2 M Cu: 18.2 g Cu(NO₃)₂*3H₂O were dissloved in 38 cc d.i. H₂O

2 M Zn: 11.0 g Zn(NO₃)₂*6H₂O were dissloved in 19 cc d.i. H₂O

2 M Mn: 2.3 g Mn(NO₃)₂*xH₂O were dissloved in 7 cc d.i. H₂O

2 M Na₂CO₃: 47.0 g Na₂CO₃*H₂O were dissloved in 188 cc d.i. H₂O

A 2 liter flask was filled with a slurry made of 10 g SAPO-41 (OLE 380 from UOP) and 500 ml d.i. H₂O. The slurry was heated to 70° C. and stirred. The Cu, Zn, and Mn solutions were mixed to form a solution 1. The Na₂CO₃ solution was labeled as solution 2. While stirring the slurry and maintaining its temperature at 70° C., solutions 1 and 2 were added simultaneously to the water, with pH control during addition at 7.0. The reaction was considered complete when all of solution 1 had been added. The mixture was then kept at temperature and under stirring for another hour. The stirring was stopped and the mixture was cooled to room temperature. The precipitate was then filtered and thoroughly washed with warm d.i. H₂O (1500-2000 cc H₂O). The precipitate was finally dried overnight at 85° C.

The precipitate was calcined under air according to the following schedule:

1) 2 hours ramp from room temperature to 150° C.

2) 0.5 hour at 150° C.

3) 2 hours ramp from 150° C. to 350° C.

4) 3 hours at 350° C., then heating stopped

5) cool down to room temperature

### Example 5

This example describes the preparation of a methanol and dimethyl ether forming catalyst (E) having the following composition:

33% MCM-49/67% (60% CuO/30% ZnO/10% Mn₂O₃ (nominal)

The following solutions were prepared:

2 M Cu: 18.2 g Cu(NO₃)₂*3H₂O were dissloved in 38 cc d.i. H₂O

2 M Zn: 11.0 g Zn(NO₃)₂*6H₂O were dissloved in 19 cc d.i. H₂O

2 M Mn: 2.3 g Mn(NO₃)₂*xH₂O were dissloved in 7 cc d.i. H₂O

2 M Na₂CO₃: 93.0 g Na₂CO₃*H₂O were dissloved in 375 cc d.i. H₂O

A 2 liter flask was filled with a slurry made of 5 g MCM-49 and 500 ml d.i. H₂O. The slurry was heated to 70° C. and stirred. The Cu, Zn, and Mn solutions were mixed to form a solution 1. The Na₂CO₃ solution was labeled as solution 2. While stirring the slurry and maintaining its temperature at 70° C., solutions 1 and 2 were added simultaneously to the water, with pH control during addition at 7.0. The reaction was considered complete when all of solution 1 had been added. The mixture was then kept at temperature and under stirring for another hour. The stirring was stopped and the mixture was cooled to room temperature. The precipitate was then filtered and thoroughly washed with warm d.i. H₂O (1500-2000 cc H₂O). The precipitate was finally dried overnight at 85° C.

The precipitate was calcined under air according to the following schedule:

1) 2 hours ramp from room temperature to 150° C.

2) 0.5 hour at 150° C.

3) 2 hours ramp from 150° C. to 350° C.

4) 3 hours at 350° C., then heating stopped

5) cool down to room temperature

### Example 6 (Comparative)

2 ml of catalyst A were loaded in a reactor and tested for syngas conversion under the following conditions: T=300° C., P=750 psi, GHSV=5000, feed composition: 60% H₂, 25% CO, 5% CO₂, 10% N₂. Average conversions and selectivities over an 18-hour period were as follows:

COx conversion=47%

Methanol selectivity=7.1%

Methane selectivity=0.5%

DME selectivity=90.2%

Other C₂s, C₃s, C₄s, and C₅+=balance

As indicated, the catalyst demonstrated a dimethyl ether (DME) selectivity >90%.

### Example 7 (Comparative)

2 ml of catalyst B were loaded in a reactor and tested for syngas conversion under the following conditions: T=300° C., P=750 psi, GHSV=5000, feed composition: 60% H₂, 25% CO, 5% CO₂, 10% N₂. Average conversions and selectivities over a 24-hour period were as follows:

COx conversion=20.4%

Methanol selectivity=77.2%

Methane selectivity=1.2%

DME selectivity=1.4%

Other C₂s, C₃s, C₄s, and C₅+=balance

As indicated, the catalyst formed only a very small amount of DME.

### Example 8

2 ml of catalyst C were loaded in a reactor and tested for syngas conversion under the following conditions: T=300° C., P=750 psi, GHSV=5000, feed composition: 60% H₂, 25% CO, 5% CO₂, 10% N₂. Average conversions and selectivities over a 20-hour period were as follows:

COx conversion=2.1%

Methanol selectivity=63.1%

Methane selectivity=0%

DME selectivity=7.8%

Other C₂s, C₃s, C₄s, and C₅+=balance

As indicated, the amount of methanol remains the major product from the syngas conversion, but ca. 8% DME is also obtained in the product.

### Example 9

2 ml of catalyst D were loaded in a reactor and tested for syngas conversion under the following conditions: T=300° C., P=750 psi, GHSV=5000, feed composition: 60% H₂, 25% CO, 5% CO₂, 10% N₂. Average conversions and selectivities over a 22-hour period were as follows:

COx conversion=12.1%

Methanol selectivity=71.5%

Methane selectivity=0.9%

DME selectivity=16.5%

Other C₂s, C₃s, C₄s, and C₅+=balance

As indicated, methanol remains the major product from the syngas conversion, but ca. 17% DME is also obtained in the product.

### Example 10

2 ml of catalyst E were loaded in a reactor and tested for syngas conversion under the following conditions: T=300° C., P=750 psi, GHSV=5000, feed composition: 42.5% H₂, 42.5% CO, 5% CO₂, 10% N₂. Average conversions and selectivities over a 20-hour period were as follows:

COx conversion=11.2%

Methanol selectivity=62.3%

Methane selectivity=0.4%

DME selectivity=22.3%

Other C₂s, C₃s, C₄s, and C₅+=balance

As indicated, methanol remains the major product from the syngas conversion, but ca. 22% DME is obtained in the product.

TABLE 1

| | CATALYST | | | | |
|---|---|---|---|---|---|
| | A | B | C | D | E |
| | (Ex. 6) | (Ex. 7) | (Ex. 8) | (Ex. 9) | (Ex. 10) |
| Loading (ml) | 2 | 2 | 2 | 2 | 2 |
| Temperature (C.) | 300 | 300 | 300 | 300 | 300 |
| Pressure (psi) | 750 | 750 | 750 | 750 | 750 |
| GHSV | 5000 | 5000 | 5000 | 5000 | 5000 |
| CO/H₂/CO₂/N₂ (%) | 60/25/5/10 | 60/25/5/10 | 60/25/5/10 | 60/25/5/10 | 60/25/5/10 |
| COₓ conversion (%) | 47 | 20.4 | 2.1 | 12.1 | 17.7 |
| MeOH selectivity (%) | 7.1 | 77.2 | 63.1 | 71.5 | 76.5 |
| CH₄ selectivity (%) | 0.5 | 1.2 | 0 | 0.9 | 0.5 |
| DME selectivity (%) | 90.2 | 1.4 | 7.8 | 16.5 | 13.7 |

Catalysts C-E produce a favorable product balance of MeOH and DME without any significant increase of CH₄ production. Preferably, catalysts of this type produce a methanol and DME containing product having from 50 wt % to 95 wt % methanol and from 5 wt % to 25 wt % DME, more preferably from 60 wt % to 90 wt % methanol and from 7 wt % to 20 wt % DME.

The principles and modes of operation of this invention have been described above with reference to various exemplary and preferred embodiments. As understood by those of skill in the art, the overall invention, as defined by the claims, encompasses other preferred embodiments not specifically enumerated herein.

What is claimed is:

1. A methanol and dimethyl ether forming catalyst comprising a mixture of at least two catalyst components, wherein a first of the catalyst components includes at least one oxide of at least one element selected from the group consisting of copper, silver, zinc, boron, magnesium, aluminum, vanadium, chromium, manganese, gallium, palladium, osmium, and zirconium, and a second of the catalyst components includes at least one MCM or SAPO molecular sieve.

2. The catalyst of claim 1, wherein the first of the catalyst components is a copper-based catalyst that includes at least one oxide of at least one element selected from the group consisting of silver, zinc, boron, magnesium, aluminum, vanadium, chromium, manganese, gallium, palladium, osmium, and zirconium.

3. The catalyst of claim 1, wherein the first of the catalyst components is a copper-based catalyst that includes at least one oxide of at least one element selected from the group consisting of zinc, magnesium, aluminum, chromium, manganese, and zirconium.

4. The catalyst of claim 1, wherein the first of the catalyst components is a copper-based catalyst that includes zinc oxide, manganese oxide, or a combination thereof.

5. The catalyst of claim 1, wherein the molecular sieve is an intermediate or large pore MCM or SAPO molecular sieve.

6. The catalyst of claim 1, wherein the molecular sieve is an intermediate pore MCM or SAPO molecular sieve.

7. The catalyst of claim 1, wherein the second of the catalyst components includes at least one MCM or SAPO molecular sieve selected from the group consisting of MCM-22, MCM-36, MCM-49, MCM-56, MCM-68, SAPO-11, SAPO-31, and SAPO-41.

8. The catalyst of claim 1, wherein the second of the catalyst components includes at least one MCM or SAPO molecular sieve selected from the group consisting of MCM-49, SAPO-11, and SAPO-41.

9. The catalyst of claim 8, wherein the first of the catalyst components is a copper-based catalyst that includes at least one oxide of at least one element selected from the group consisting of zinc, magnesium, aluminum, chromium, manganese, and zirconium.

10. The catalyst of claim 8, wherein the first of the catalyst components is a copper-based catalyst that includes zinc oxide, manganese oxide, or a combination thereof.

11. A method of making a methanol and dimethyl ether forming catalyst comprising:
mixing together a liquid and at least a first and second catalyst component to form a slurry, wherein the first catalyst component contains at least one oxide of at least one element selected from the group consisting of copper, silver, zinc, boron, magnesium, aluminum, vanadium, chromium, manganese, gallium, palladium, osmium, and zirconium, and the second catalyst component contains at least one MCM or SAPO molecular sieve; and
drying the slurry to form the methanol and dimethyl ether forming catalyst.

12. The method of claim 11, wherein the first of the catalyst components is a copper-based catalyst that includes at least one oxide of at least one element selected from the group consisting of silver, zinc, boron, magnesium, aluminum, vanadium, chromium, manganese, gallium, palladium, osmium, and zirconium.

13. The method of claim 11, wherein the first of the catalyst components is a copper-based catalyst that includes at least one oxide of at least one element selected from the group consisting of zinc, magnesium, aluminum, chromium, manganese, and zirconium.

14. The method of claim 11, wherein the first of the catalyst components is a copper-based catalyst that includes zinc oxide, manganese oxide, or a combination thereof.

15. The method of claim 11, wherein the molecular sieve is an intermediate or large pore MCM or SAPO molecular sieve.

16. The method of claim 11, wherein the molecular sieve is an intermediate pore MCM or SAPO molecular sieve.

17. The method of claim 11, wherein the second of the catalyst components includes at least one MCM or SAPO molecular sieve selected from the group consisting of MCM-22, MCM-36, MCM-49, MCM-56, MCM-68, SAPO-11, SAPO-31, and SAPO-41.

18. The method of claim 11, wherein the second of the catalyst components includes at least one MCM or SAPO molecular sieve selected from the group consisting of MCM-49, SAPO-11, and SAPO-41.

19. The method of claim 18, wherein the first of the catalyst components is a copper-based catalyst that includes at least one oxide of at least one element selected from the group consisting of zinc, magnesium, aluminum, chromium, manganese, and zirconium.

20. The method of claim 18, wherein the first of the catalyst components is a copper-based catalyst that includes zinc oxide, manganese oxide, or a combination thereof.

21. A process for producing olefin product from syngas, comprising:
contacting a methanol and dimethyl ether forming catalyst with syngas to form a composition containing methanol and dimethyl ether, wherein the catalyst comprises a first catalyst component that includes an oxide of at least one element selected from the group consisting of copper, silver, zinc, boron, magnesium, aluminum, vanadium, chromium, manganese, gallium, palladium, osmium, and zirconium, and a second catalyst component that includes at least one MCM or SAPO molecular sieve; and
contacting the composition with an olefin forming catalyst to form the olefin product.

22. The process of claim 21, wherein the first of the catalyst components is a copper-based catalyst that includes at least one oxide of at least one element selected from the group consisting of silver, zinc, boron, magnesium, aluminum, vanadium, chromium, manganese, gallium, palladium, osmium, and zirconium.

23. The process of claim 21, wherein the first of the catalyst components is a copper-based catalyst that includes at least one oxide of at least one element selected from the group consisting of zinc, magnesium, aluminum, chromium, manganese, and zirconium.

24. The process of claim 21, wherein the first of the catalyst components is a copper-based catalyst that includes zinc oxide, manganese oxide, or a combination thereof.

25. The process of claim 21, wherein the molecular sieve is an intermediate or large pore MCM or SAPO molecular sieve.

26. The process of claim 21, wherein the molecular sieve is an intermediate pore MCM or SAPO molecular sieve.

27. The process of claim 21, wherein the second of the catalyst components includes at least one MCM or SAPO molecular sieve selected from the group consisting of MCM-22, MCM-36, MCM-49, MCM-56, MCM-68, SAPO-11, SAPO-31, and SAPO-41.

28. The process of claim 21, wherein the second of the catalyst components includes at least one MCM or SAPO molecular sieve selected from the group consisting of MCM-49, SAPO-11, and SAPO-41.

29. The process of claim 28, wherein the first of the catalyst components is a copper-based catalyst that includes at least one oxide of at least one element selected from the group consisting of zinc, magnesium, aluminum, chromium, manganese, and zirconium.

30. The process of claim 28, wherein the first of the catalyst components is a copper-based catalyst that includes zinc oxide, manganese oxide, or a combination thereof.
